(19) United States
(12) Patent Application Publication
(10) Pub. No.: US 2023/0124720 A1
(43) Pub. Date: Apr. 20, 2023

(54) APPLICATION OF ACRIDINEDIONE COMPOUND IN PREPARATION OF ANTI-DIABETIC DRUGS

(71) Applicant: THE FOURTH MILITARY MEDICAL UNIVERSITY, Xi'an (CN)

(72) Inventors: Xueying LIU, Xi'an (CN); Xin SHEN, Xi'an (CN); Jiyuan LIU, Xi'an (CN); Shengyong ZHANG, Xi'an (CN); Qingwei WANG, Xi'an (CN); Jie ZHANG, Xi'an (CN); Zhao WEI, Xi'an (CN); Dongxu ZHANG, Xi'an (CN); Jialong LIANG, Xi'an (CN); Xinlei ZHANG, Xi'an, Shaanxi (CN); Shijie JU, Xi'an, Shaanxi (CN)

(21) Appl. No.: 17/956,867

(22) Filed: Sep. 30, 2022

Related U.S. Application Data
(63) Continuation of application No. PCT/CN2021/085944, filed on Apr. 8, 2021.

(30) Foreign Application Priority Data
Apr. 8, 2020 (CN) 202010277712.3

(51) Int. Cl. A61K 31/473; A61P 3/10

(52) U.S. Cl. CPC A61K 31/473; A61P 3/10

(57) ABSTRACT

The present application provides the use of an acridinedione compound in the preparation of an antidiabetic medicament, which belongs to the field of biomedical technology. The present application demonstrates for the first time that the acridinedione compound can improve insulin resistance by activating and upregulating GPR40 protein expression, participating in GPR40-PPARγ-PI3K/Akt-GLUT4 signaling pathway, promoting insulin secretion and increasing glucose consumption in liver and muscle tissue. The target of the acridinedione compound is the GPR40 receptor, its insulinotropic effect is glucose-dependent, and its hypoglycemic effect disappears when peripheral blood glucose falls below a certain level. The preparation of the acridinedione compound into an antidiabetic medicaments will provide entirely new options and strategies for the treatment of diabetes.

### TECHNICAL FIELD

The present application belongs to the field of biomedical technology, and in particular relates to the use of an acridinedione compound in the preparation of an antidiabetic medicament.

### BACKGROUND

Currently about 463 million adults between 20 and 79 years of age worldwide suffer from diabetes, with the number of Type 2 diabetes (T2DM) patients accounting for over 90% of the total number of diabetic patients. The number of deaths due to diabetes mellitus and its complications was statistically as high as 4.2 million in 2019. Chinese diabetics have reached 116 million, accounting for about one-fourth of the total number of diabetics worldwide, and the number of deaths due to diabetes and its complications in our country in 2019 was about 823 thousand. Type 2 diabetes has become a significant problem affecting human health. Traditional anti-type 2 diabetes drugs exert hypoglycemic effects primarily by stimulating insulin secretion from pancreatic islet β-cells and increasing insulin sensitivity, which-are independent of peripheral blood glucose, thus lowering blood glucose with these drugs increases the risk of hypoglycemia in patients. GPR40, a member of the G protein-coupled receptor family, is a fatty acid-specific receptor distributed predominantly in islet β-cells, intestinal K and L cells. GPR40-mediated insulin secretion is glucose-dependent, and its hypoglycemic effect disappears when peripheral blood glucose falls below a certain level, thereby reducing the incidence of hypoglycemia. The GPR40 receptor has emerged as a potential target for the improvement of drugs against type 2 diabetes due to its unique glycemic regulating role, and the improvement of drugs against type 2 diabetes using GPR40 agonists or lead compounds has important implications.

### SUMMARY

It is an object of the present application to provide use of an acridinedione compound in the preparation of an antidiabetic medicament, providing entirely new options and strategies for the treatment of diabetes.

The present application is achieved by the following technical solutions:

An acridinedione compound, or a pharmaceutically acceptable salt thereof, or a pharmaceutically acceptable ester thereof, the compound having the following structure:

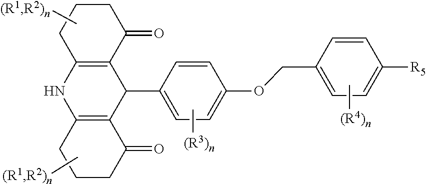

R¹ and R² are each independently hydrogen, C1-C3 alkyl, —COOH, —CH₂Ph, —CH₂CH(CH₃)₂, -Ph, or

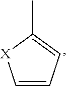

where X=O, N or S;

R³ is hydrogen, halogen, —CF₃, C1-C4 alkyl or alkoxy, —NO₂, or —OH;

R⁴ is hydrogen, halogen, —CF₃, C1-C3 alkyl or alkoxy, —NO₂, or —OH;

R⁵ is —COOH, —COOCH₃ or —COOC₂H₅; and

n is 0, 1 or 2.

Use of an acridinedione compound or a pharmaceutically acceptable salt or a pharmaceutically acceptable ester thereof in the preparation of an antidiabetic medicament, wherein the acridinedione compound has the following structure:

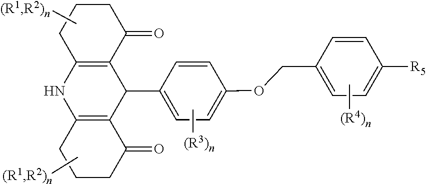

R¹ and R² are each independently hydrogen, C1-C3 alkyl, —COOH, —CH₂Ph, —CH₂CH(CH₃)₂, -Ph, or

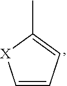

where X=O, N or S;

R³ is hydrogen, halogen, —CF₃, C1-C4 alkyl or alkoxy, —NO₂, or —OH;

R⁴ is hydrogen, halogen, —CF₃, C1-C3 alkyl or alkoxy, —NO₂, or —OH;

R⁵ is —COOH, —COOCH₃ or —COOC₂H₅; and

n is 0, 1 or 2.

A further improvement of the application is that the antidiabetic medicament is a GPR40 agonist.

A further improvement of the present application is that the antidiabetic medicament is a glucose-dependent insulinotropic drug.

A further improvement of the present application is that the antidiabetic medicament is a clinically acceptable pharmaceutical formulation.

A further improvement of the application is that the pharmaceutical formulation is a tablet, capsule, granule or injection.

Use of an acridinedione compound or a pharmaceutically acceptable salt thereof or a pharmaceutically acceptable ester thereof in the preparation of a GPR40 agonist, wherein the compound has the following structure:

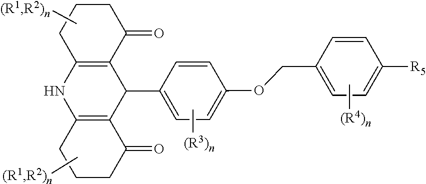

R¹ and R² are hydrogen, C1-C3 alkyl, —COOH, —CH₂Ph, —CH₂CH(CH₃)₂, -Ph, or

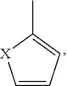

where X=O, N or S;

R³ is hydrogen, halogen, —CF₃, C1-C4 alkyl or alkoxy, —NO₂, or —OH;

R⁴ is hydrogen, halogen, —CF₃, C1-C3 alkyl or alkoxy, —NO₂, or —OH;

R⁵ is —COOH, —COOCH₃ or —COOC₂H₅; and

n is 0, 1 or 2.

Compared with the prior art, the present application has the following beneficial technical effects.

The present application has been found for the first time that an acridinedione compound or a pharmaceutically acceptable salt thereof can be used in the preparation of an antidiabetic medicament, and confirmed that the acridinedione compound can exert an action against type 2 diabetes by activating and upregulating GPR40 protein expression, participating in GPR40-PPARγ-PI3K/Akt-GLUT4 signaling pathway, promoting insulin secretion, increasing glucose consumption in liver and muscle tissue, improving insulin resistance. The acridinedione compound act as a target at the GPR40 receptor, and its insulinotropic effect is glucose-dependent, and its hypoglycemic effect disappears when peripheral blood glucose falls below a certain level. The preparation of the acridinedione compound as antidiabetic medicaments would provide entirely new options and strategies for the treatment of diabetes.

### BRIEF DESCRIPTION OF DRAWINGS

FIG. 1 shows that ADD-16 promotes glucose-stimulated insulin secretion in MIN6 cells, where A is cytotoxicity, including 6 h, 12 h, 24 h and 48 h; B is insulin secretion. x̅±s (n=6), *P<0.05, **P<0.01, ***P<0.001 vs. Con group.

FIG. 2 shows the effect of ADD-16 on STZ-induced blood glucose regulation in T2DM rats, where A is the postprandial blood glucose change trend; B is the postprandial blood glucose value of each group of rats at the end of the experiment; C is the glycated hemoglobin value. x̅±s (n=10), *P<0.05, **P<0.01, ***P<0.001 vs. Con group; #P<0.05, ##P<0.01, ###P<0.001 vs. TD group.

FIG. 3 shows the effect of ADD-16 on STZ-induced glucose tolerance in T2DM rats, where A is the OGTT curve; B is the area under the curve. x̅±s (n=10), *P<0.05, **P<0.01, ***P<0.001 vs. Con group; #P<0.05, ##P<0.01, ###P<0.001 vs. TD group.

FIG. 4 shows that ADD-16 ameliorates insulin resistance in STZ-induced T2DM rats, where A is serum insulin level; B is the index of insulin resistance. x̅±s (n=10), *P<0.05, **P<0.01, ***P<0.001 vs. Con group; #P<0.05, ##P<0.01, ###P<0.001 vs. TD group.

FIG. 5 shows the effect of ADD-16 on STZ-induced insulin tolerance in T2DM rats, where A is the ITT curve; B is the area under the curve. x̅±s (n=10), *P<0.05, **P<0.01, ***P<0.001 vs. Con group; #P<0.05, #P<0.01, ###P<0.001 vs. TD group.

FIG. 6 shows the effect of ADD-16 on STZ-induced fat metabolism in T2DM rats, where A is FFA; B is TG; C is TC; D is LDL; E is the HDL content. x̅±s (n=10), *P<0.05, **P<0.01, ***P<0.001 vs. Con group; #P<0.05, ##P<0.01, ###P<0.001 vs. TD group.

FIG. 7 is a plasma concentration-time profile (x̅±s, n=8) following a single oral administration of ADD-16 in rats.

FIG. 8 shows tissue concentration profiles (x̅±s, n=6) at various time points after a single oral administration of ADD-16 in rats.

FIG. 9 shows that ADD-16 ameliorates MIN6 cell insulin resistance through GPR40, where A is insulin secretion from each group after 24 h of group treatment; B is the change in insulin secretion level under different dosing conditions. x̅±s (n=6), *P<0.05, **P<0.01, ***P<0.001 vs. Con group; #P<0.05, ##P<0.01, ###P<0.001 vs. IR group.

FIG. 10 shows the effect of ADD-16 on insulin signaling related molecule expression in ZDF rat islet tissue. x̅±s (n=3), *P<0.05, **P<0.01, ***P<0.001 vs. Con group; #P<0.05, ##P<0.01, ###P<0.001 vs. TD group.

FIG. 11 shows the effect of ADD-16 on insulin signaling related molecule expression in MIN6, where A is the WB outcome in MIN6 cells; B is the immunofluorescence staining result of GPR40 in MIN6 cells. x̅±s (n=3), *P<0.05, **P<0.01, ***P<0.001 vs. Con group; #P<0.05, ##P<0.01, ###P<0.001 vs. IR group.

FIG. 12 shows the conformational overlap pattern of 32 compounds binding GPR40.

### DESCRIPTION OF EMBODIMENTS

The application will now be described in further detail with reference to specific examples, which are intended to be illustrative, but not restrictive, of the present application.

Use of an acridinedione compound or a pharmaceutically acceptable salt thereof or a pharmaceutically acceptable ester thereof in the preparation of an antidiabetic medicament, wherein the acridinedione compound has the structure shown below.

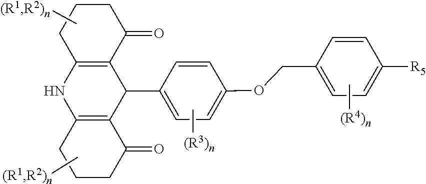

R¹ and R² are each independently hydrogen, C1-C3 alkyl, —COOH, —CH₂Ph, —CH₂CH(CH₃)₂, -Ph, or

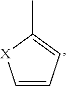

where X=O, N or S;

R³ is hydrogen, halogen, —CF₃, C1-C4 alkyl or alkoxy, —NO₂, or —OH;

R⁴ is hydrogen, halogen, —CF₃, C1-C3 alkyl or alkoxy, —NO₂, or —OH;

R⁵ is —COOH, —COOCH₃ or —COOC₂H₅; and

n is 0, 1 or 2.

Preferably, the antidiabetic medicament is a GPR40 agonist.

Preferably, the antidiabetic medicament is a glucose-dependent insulinotropic drug.

Preferably, the antidiabetic medicament is a clinically acceptable pharmaceutical formulation.

Preferably, the pharmaceutical formulation is other oral dosage forms such as tablets, capsules, granules or injectable dosage forms.

Use of an acridinedione compound or a pharmaceutically acceptable salt thereof or a pharmaceutically acceptable ester thereof in the preparation of a GPR40 agonist.

The pharmaceutically acceptable salt of the acridinedione compound is potassium, sodium or calcium carboxylates, i.e., R5 is potassium carboxylate, sodium carboxylate or calcium carboxylate, methyl carboxylate or ethyl carboxylate.

### 1. Synthesis of Acridinedione GPR40 Agonist

Taking the synthesis of Add-16, Add-17, Add-18 and Add-19 as an example, the synthetic route is as follows:

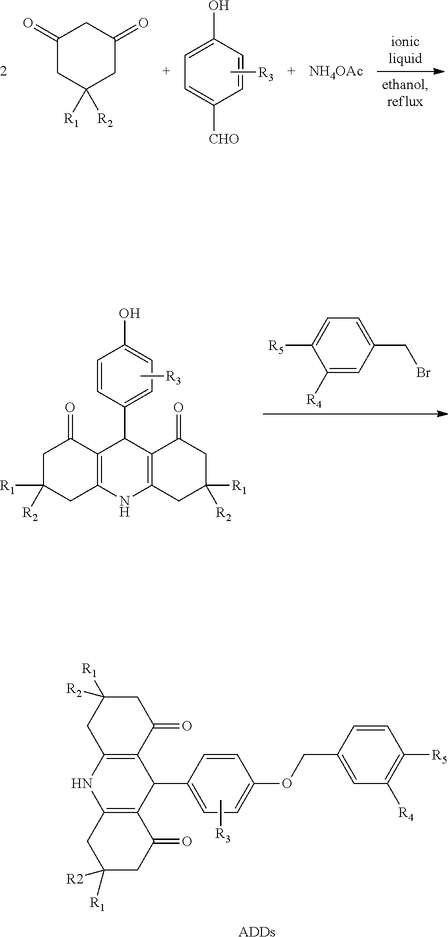

ADD-16 synthesis taken as an example.

5,5-dimethyl-1,3-cyclohexanedione (2.8 g, 20 mmol), 3-bromo-4-hydroxybenzaldehyde (2.01 g, 10 mmol), and ammonium acetate (2.31 g, 30 mmol) were placed in a 100 mL volumetric flask, and an unpurified ionic liquid catalyst (0.2 g, 0.36 mmol) was added and refluxed at 80° C. for 4 h. The reaction was monitored by TLC, the temperature was reduced to room temperature after completion of the reaction, followed by filtering, washing with anhydrous ethanol, and drying to give 3.79 g of a crude acridinedione intermediate, with a yield of about 83.3, which was used in the next step without purification.

50 mL of DMSO was added to a round bottom flask, and KOH (3.06 g, 0.055 mol) was ground to powder under an infrared lamp and added and stirred for 10 min. The prepared acridinedione intermediate (3.79 g, 9.1 mmol) was added and stirred for 15 min, and then p-bromomethylbenzoic acid (1.96 g, 9.1 mmol) was added for reaction at room temperature for 4 h. The reaction was monitored by TLC. 50 mL of distilled water was added after the completion of the reaction, and pH was adjusted to 5.0 by concentrated hydrochloric acid.

Extraction was performed with EtOAc, organic layers were combined, dried over anhydrous Na₂SO₄ overnight, and purified by flash column chromatography (petroleum ether:ethyl acetate/1:9 V:V) to give a light yellow solid 2.74 g, with a yield of approximately 52.1%.

ADD-16 (R¹═R²═CH₃, m-R³═Br, R⁴═H, R⁵═COOH, n=1), nuclear magnetic data ¹H NMR (400 MHz, DMSO-d₆) δ9.323 (s, 1H), 7.963 (d, J=8.4 Hz, 2H), 7.559 (d, J=8.4 Hz, 2H), 7.336 (d, J=2.0 Hz, 1H), 7.088 (dd, J=2.0, 2.0 Hz, 1H), 7.007 (dd, J=8.3, 8.8 Hz, 1H), 4.67 (s, 1H), 2.453 (d, J=16.8 Hz, 2H), 2.345 (d, J=16.8 Hz, 2H), 2.181 (d, J=16.0 Hz, 2H), 2.001 (d, J=16.0 Hz, 3H), 1.014 (s, 6H), 0.8886 (s, 6H); ¹³C NMR (126 MHz, DMSO-d₆) δ194.89, 167.53, 152.60, 149.89, 142.26, 142.00, 132.54, 130.72, 129.92, 128.29, 113.93, 111.58, 110.68, 79.94, 69.90, 50.64, 32.63, 29.52, 26.92. High resolution mass spectral data HRMS (ESI⁺) for C₃₁H₃₂BrNO₅[M+H]⁺: m/z cale., 578.1537; found, 578.1529.

ADD-17 (R¹═R²═CH₃, m-R³═Cl, R⁴═H, R⁵═COOH, n=1) was synthesized in the same way, nuclear magnetic data ¹H NMR (400 MHz, DMSO-d₆), nuclear magnetic data: ¹H NMR (400 MHz, DMSO-d₆) δ9.292 (s, 1H), 7.963 (d, J=8.3 Hz, 2H), 7.559 (d, J=8.5 Hz, 2H), 7.183 (d, J=2.1 Hz, 1H), 6.988 (dd, J=2.0, 2.0 Hz, 1H), 6.847 (dd, J=8.3, 8.8 Hz, 1H), 4.69 (s, 1H), 2.443 (d, J=17.8 Hz, 2H), 2.315 (d, J=17.0 Hz, 2H), 2.171 (d, J=16.1 Hz, 2H), 1.991 (d, J=16.2 Hz, 2H), 1.004 (s, 6H), 0.8786 (s, 6H); ¹³C NMR (126 MHz, DMSO-d₆) δ194.49, 167.03, 152.10, 149.49, 141.86, 141.50, 132.04, 130.72, 129.92, 128.29, 113.43, 111.08, 110.08, 79.94, 69.90, 52.64, 32.13, 29.02, 26.52. High resolution mass spectral data HRMS (ESI⁺)for C₃₁H₃₂ClNO₅[M+H]⁺: m/z cale., 533.1969; found, 533.1959.

ADD-18 R¹═R²═CH₃, m-R³═CF₃, R⁴═H, R⁵═COOH, n=1) was synthesized in the same way, nuclear magnetic data: ¹H NMR (400 MHz, DMSO-d₆) δ9.523 (s, 1H), 8.163 (d, J=8.2 Hz, 2H), 8.129 (d, J=8.0 Hz, 2H), 7.666 (d, J=2.2 Hz, 1H), 7.498 (dd, J=2.1, 2.0 Hz, 1H), 7.157 (dd, J=8.5, 8.9 Hz, 1H), 4.79 (s, 1H), 2.433 (d, J=16.5 Hz, 2H), 2.345 (d, J=16.6 Hz, 2H), 2.081 (d, J=16.1 Hz, 2H), 2.011 (d, J=16.0 Hz, 2H), 1.024 (s, 6H), 0.8986 (s, 6H); ¹³C NMR (126 MHz, DMSO-d₆) δ194.19, 169.53, 153.60, 150.89, 144.26, 143.00, 134.54, 132.72, 130.92, 129.29, 114.93, 112.58, 111.68, 80.94, 70.90, 50.84, 32.83, 29.72, 27.32. High resolution mass spectral data HRMS (ER⁺) for C₃₂H₃₂F₃NO₅[M+H]⁺: m/z cale., 567.2233; found, 567.2229.

ADD-19 (R¹═R²═CH₃, o-R³═Br, R⁴═H , R⁵═COOH, n=1) was synthesized in the same way, nuclear magnetic data: ¹H NMR (400 MHz, DMSO-d₆)δ9.223 (s, 1H), 8.143 (d, J=8.2 Hz, 2H), 8.129 (d, J=8.0 Hz, 2H), 7.496 (d, J=2.2 Hz, 1H), 7.338 (dd, J=2.1, 2.0 Hz, 1H), 7.307 (dd, J=8.5, 8.9 Hz, 1H), 4.78 (s, 1H), 2.433 (d, J=16.5 Hz, 2H), 2.345 (d, J=16.6 Hz, 2H), 2.081 (d, J=16.1 Hz, 2H), 2.011 (d, J=16.0 Hz, 2H), 1.024 (s, 6H), 0.8986 (s, 6H); ¹³C NMR (126 MHz, DMSO-d₆)δ195.19, 168.53, 154.60, 151.89, 143.26, 142.00, 133.54, 131.72, 130.92, 129.29, 114.93, 112.58, 111.68, 80.94, 70.90, 50.84, 32.83, 29.72, 27.32. High resolution mass spectral data HRMS (ESI⁺) for C₃₂H₃₂F₃NO₅[M+H]⁺: m/z cale., 567.2233; found, 567.2230.

### 2. GPR40 Agonistic Activity Assay

2.1 Methods: After the confluence degree of the HEK-293T cells stably transfected with the GPR40 expression reached 80%, the cell concentration was adjusted to be seeded at a density of 1×10⁵/mL in a 96-well plate at 100 μL per well and incubated for 24 h at 37° C. in a 5% CO₂ incubator; after gentle washing with HBSS buffer per well, 100 μL of a Fluo-4 AM dye solution was added and incubated for 30 min at 37° C. in dark place, wherein the dye Fluo-4 AM was made up in HBSS containing 0.1 BSA, 20 mmol/L HEPES and 2.5 mmol/L probenecid with a final concentration of 3 μmol/L; at the end of incubation, excess dye was washed with HBSS containing 0.1 BSA, 20 mmol/L HEPES and 2.5 mmol/L probenecid and equilibrated with HBSS for 10 min; compounds of different concentration gradients, as well as agonist positive control GW9508 (1 μmol/L), blocker positive control GW1100 (10 μmol/L), negative control DMSO (0.1% final concentration) and blank (without any additives) were added. There were triplicate wells for each group, and a Flexstation instrument was used for detecting and reading fluorescence values. Each compound dilution concentration was: 200 μmol/L, 100 μmol/L, 50 μmol/L, 10 μmol/L, 5 μmol/L.

EC50 value for each compound was calculated from the measured fluorescence value according to the following formula:

Relative agonism ratio=(fluorescence value of test compound-fluorescence value of negative control)/(fluorescence value of agonist positive control-fluorescence value of negative control)*100%;

Inhibition ratio=(fluorescence value of negative control-fluorescence value of test compound)/(fluorescence value of negative control-fluorescence value of blocker positive control)*100%.

### 2.2 Results

The candidate compounds screened were evaluated using HEK-293T cells stably overexpressing GPR40. ADD-16 was found to have the highest agonistic activity, which was comparable to the GPR40 endogenous agonist Palmitic acid (PA), and was therefore selected to proceed with a focused evaluation of pharmacopharmacodynamics. Agonistic activity of specific candidate compounds is shown in Table 3.

### 3. Effect of ADD-16 on Glucose-Stimulated Insulin Secretion in MIN6 Cells (GSIS Experiments)

### 3.1 Methods

MIN6 cells were seeded in a 24-well plate at an appropriate concentration, and after continued culture until cell confluence exceeded 80%, administration was carried out in the following groups: normal control group (Con), ADD group (ADD-16 was administered at a concentration of 100 μmol/L, 30 μmol/L, 10 μmol/L, 3 μmol/L, 1 μmol/L, 0.3 μmol/L, respectively), TAK875 group (administered at a concentration of 10 μmol/L, 3 μmol/L, 1 μmol/L, respectively), with 6 replicates per group. Incubation was continued for 24 h after group dosing, media was aspirated and gently rinsed 2 times with a sugar-free KRB buffer. After rinsing, the sugar-free KRB buffer was added and incubated at 37° C. for 10 min. At the end of the incubation, the buffer was aspirated and each well was exhausted as much as possible. Each group was dosed with 16.7 mmol/L glucose in a KRB buffer and incubated at 37° C. for 1 hour. All media in the wells was aspirated after 1 h, centrifuged at 2000 rmp/min for 20 min, and the supernatant was collected and stored at −20° C. The insulin content was measured for each group following the instructions of the mouse insulinase ELISA assay kit.

Insulinase ELISA assay: the supernatant of each set of media was taken and processed according to the procedure of Table 1:

TABLE 1
Operating Parameters

| | Samples | Blank wells |
|---|---|---|
| Samples | 10 μL | — |
| Sample Dilution | 40 μL | — |
| Enzyme Labeling Reagent | 50 μL | — |

mixing well, sealing with plate blocking membrane, incubating at
37° C. for 30 min, rinsing 5 times with washing solution, tap drying

| | | |
|---|---|---|
| Developer A | 50 μL | 50 μL |
| Developer B | 50 μL | 50 μL |

After shaking, developing color for 10 min at 37° C. in dark

| | | |
|---|---|---|
| Stop Solution | 50 μL | 50 μL |

The final mixture was mixed well with shaking and the OD was read on a microplate reader at 450 nm. The insulin content of each group was calculated.

### 3.2 Results

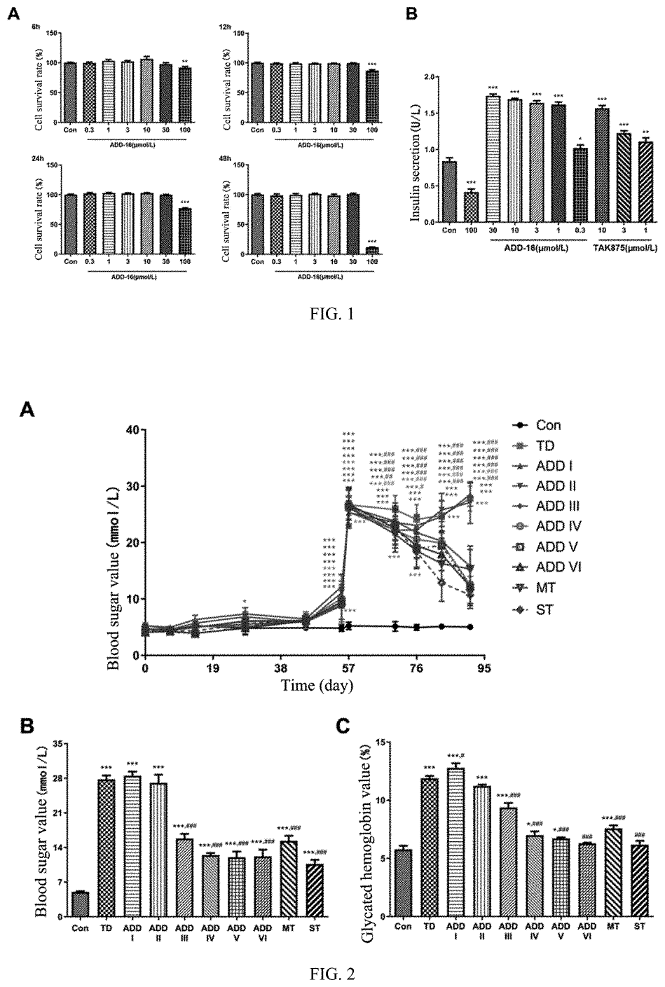

Referring to FIG. 1, from which it can be seen that ADD-16 is able to promote glucose-stimulated insulin secretion by MIN6 cells, and that this effect is concentration dependent. Whereas 100 μmol/L ADD-16 results in decreased insulin secretion compared to the Con group due to its inhibitory effect on MIN6 cell growth. Compared to the TAK875 control group, the insulinotropic effect of ADD-16 was found to be significantly better than TAK875 at the same concentrations.

### 4. Effect of ADD-16 on Glycolipid Metabolism in Type 2 Diabetic Rats

### 4.1 Methods

150 male SD rats, housed in cages in a clean constant temperature (23±2° C.) and humidity (55±10%) animal house, were manually adjusted on a daily 12 h/12 h diurnal light cycle with free access to water. After 3 days of adaptive feeding, 150 rats were randomly divided into normal and model groups. The normal (Con, n=15) group of rats remained on raw chow and the model group (n=135) of rats were given high fat high sugar chow. All rats were fed twice daily and the amount of feed was adjusted as needed to ensure adequate feed. After 8 weeks of group feeding, the model group rats were given an intraperitoneal injection of 25 mg/kg STZ solution with a 12 h fast prior to injection. After injection, feeding was continued as in the original method, blood was taken from the tail vein at 72 h for blood glucose measurement, and rats with fasting blood glucose levels ≥11.1 mmol/L on different days were screened for successful artificial induction of T2DM and administered in groups for the next experiment. 135 rats with successful modeling were randomly divided into 9 groups: (1) T2DM model group (TD, n=15): daily administration of 0.5% CMC-Na solution by gavage; (2) 0.01 mg/kg ADD-16 administration group (ADD I, n=15): daily administration of 0.01 mg/kg ADD-16 solution by gavage; (3) 0.1 mg/kg ADD-16 administration group (ADD II, n=15): daily administration of a 0.1 mg/kg ADD-16 solution by gavage; (4) 1 mg/kg ADD-16 administration group (ADD III, n=15): daily administration of a 1 mg/kg ADD-16 solution by gavage; (5) 3 mg/kg ADD-16 administration group (ADD IV, n=15): daily administration of a 3 mg/kg ADD-16 solution by gavage; (6) 10 mg/kg ADD-16 administration group (ADD V, n=15): daily administration of a 10 mg/kg ADD-16 solution by gavage; (7) 50 mg/kg ADD-16 administration group (ADD VI, n=15): daily administration of a 50 mg/kg ADD-16 solution by gavage; (8) Metformin control group (MT, n=15): daily administration of a 250 mg/kg metformin solution by gavage; (9) Sitagliptin control group (ST, n=15): daily administration of a 6 mg/kg sitagliptin solution by gavage. At the same time, the Con group was daily administrated with a 0.5% CMC-Na solution by gavage, the body weight and postprandial blood sugar (PBG) of each group of rats were monitored weekly. Relevant indicators of lipid metabolism were measured after the end of the study.

At the final week of the Oral Glucose Tolerance Test (OGTT) test, each group of rats was fasted from water 16 h after gavage administration and the fasting blood glucose value (as 0 min) was measured for each rat. A 50% glucose solution (2 g/kg) was administered by gavage according to the body weight of each rat and the blood glucose of the rats was measured at five time points, 15 min, 30 min, 60 min, 90 min, 120 min, starting from the time of administration. After the 120 min blood glucose value determination was completed, the diet was resumed.

At the last week of the Insulin Tolerance Test (ITT) test, rats in each group were fasted without water for 5 h after gavage administration, and the fasting blood glucose value (as 0 min) of each rat was measured. Insulin (0.8 U/kg) was subcutaneously injected according to the body weight of each rat, and the blood glucose of the rats was measured at four time points, 30 min, 60 min, 90 min, 120 min. After the 120 min blood glucose value determination was completed, the diet was resumed. In the test, the condition of the rats was observed, and when abnormalities occurred, the experiment was stopped in time and a glucose solution was given by gavage to prevent death due to hypoglycemia.

In T2DM rat serum insulin assay, fasting serum insulin levels in rats were measured by a commercial insulin ELISA kit. The extent of body insulin resistance was assessed using the Insulin Resistance Index, which was calculated using the Homeostasis model assessment (HOMA-IR) according to literature reports, i.e., HOMA-IR=(fasting glucose×fasting insulin)/22.5.

### 4.2 Results

### 4.2.1 Effect of ADD-16 on Blood Glucose in Artificially Induced T2DM Rats

There was a very clear trend of decreasing blood glucose in the rats in both the ADD III-ADD VI groups as well as in the MT, ST groups, with the most pronounced decrease in blood glucose in the ST group. Referring to FIG. 2, it can be seen from FIG. 2 that comparing the blood glucose values of each group of rats after the end of administration, it was found that the rats in the ADD IV, ADD V and ADD VI groups showed the greatest reduction in blood glucose, with 55.0%, 56.8% and 56.2% reduction respectively compared to the TD group, which was greater than that of the MT group (45.0%) and similar to that of the ST group (61.5%), indicating that ADD-16 produced a significant hypoglycemic effect in T2DM rats at concentrations of 3, 10, 50 mg/kg. The trend of the change in glycated haemoglobin content in each group of rats was substantially consistent with the trend of the change in postprandial blood glucose values.

### 4.2.2 ADD-16 Ameliorates Impaired Glucose Tolerance in Artificially Induced T2DM Rats

As can be seen from FIG. 3, ADD-16 was able to increase glucose tolerance in artificially induced T2DM rats.

### 4.2.3 ADD-16 Ameliorates Insulin Resistance in Artificially Induced T2DM Rats

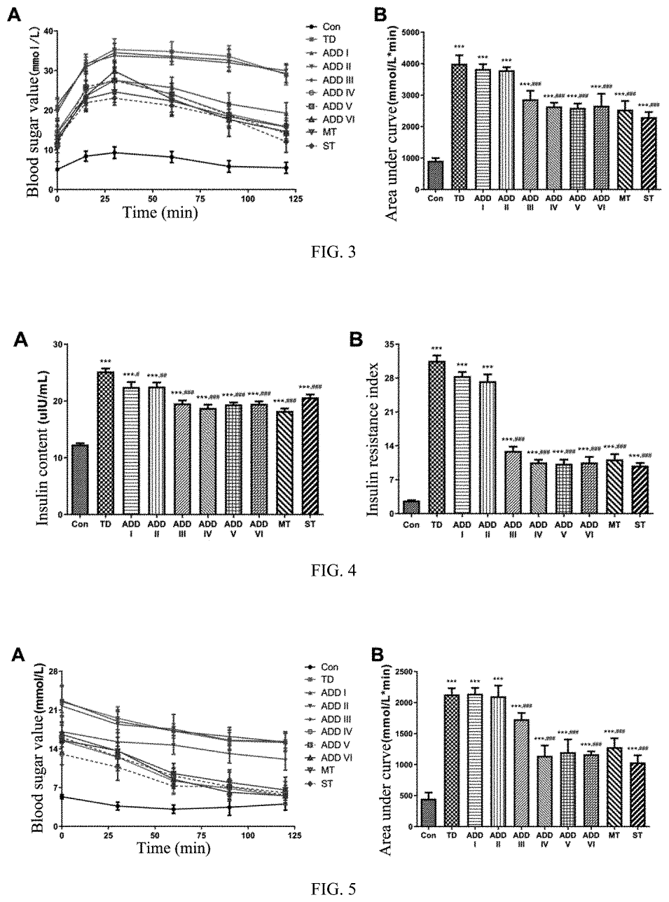

As can be seen from FIG. 4, ADD-16 was able to reduce compensatorily elevated insulin levels in T2DM rats, increase rat insulin sensitivity, while at the same time being able to promote insulin secretion to some extent, complementing the relatively undersecreted insulin levels in rats due to elevated blood glucose. Both ADD-16 and the positive control drug significantly reduced HOMA-IR values, i.e., improved insulin resistance in T2DM rats.

### 4.2.4 ADD-16 Improves Insulin Resistance in Artificially Induced T2DM Rats

As can be seen from FIG. 5, the AUC results showed a decrease of 46.5%, 43.7%, 45.2%, 39.9%, and 51.4% in the ADD IV-ADD VI groups and MT, ST groups, respectively, compared to the TD group, illustrating that ADD-16 and the positive control drug are able to improve insulin resistance in artificially induced T2DM rats.

### 4.2.5 Modulation of Artificially Induced Disturbance of Lipid Metabolism in T2DM Rats by ADD-16

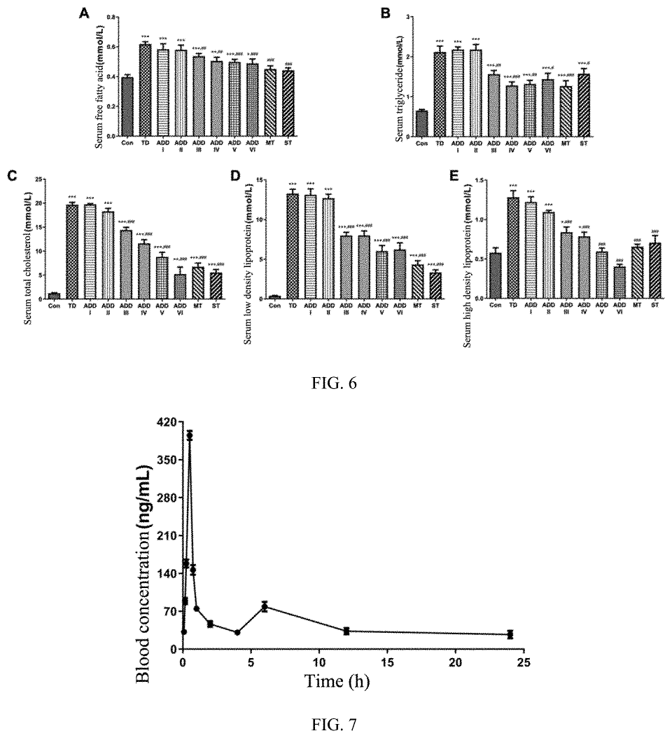

As can be seen from FIG. 6, serum FFA, TC, TG, LDL and HDL levels were significantly increased (P<0.001) in the TD group compared to the Con group, indicating that the high-fat, high-sugar diet plus STZ-induced T2DM rats developed dyslipidemia. Four weeks after administration of the intervention, serum FFA, TC, TG, LDL and HDL levels were significantly decreased in the ADD III-ADD VI group as well as in the positive control drug group, with statistically significant differences (P<0.05). Add-16 was shown to be able to ameliorate lipid metabolism disturbances in artificially induced T2DM rats. Interestingly, the TG, LDL and HDL levels of ADD V (10 mg/kg) group rats were all significantly lower than ADD IV (3 mg/kg) group rats, contrary to the trend of hypoglycemic effects of both groups, from which it can be speculated that ADD-16 has a better effect on improving lipid metabolism disorders at high concentrations, while low concentrations have a better hypoglycemic effect.

### 5. Plasma Pharmacokinetic and Histological Studies of ADD-16

### 5.1 Methods

### 5.1.1 Plasma Kinetics Studies

Eight healthy male SD rats, weighing 200-220 g, were housed in a clean animal room maintained at constant temperature (23±2° C.) and humidity (55±10%) with a 12 h/12 h artificial light cycle with free access to food and water and conditioned for 3 days prior to the experiment. All rats were fasted from water for 12 h prior to the experiment, and after oral administration of an ADD-16 solution (10 mg/kg) to each rat, and blood was taken at the orbital site at 5, 10, 15, 30, 45, 60, 120, 240, 360, 720, 1440 min. The blood was immediately fed into heparinized centrifuge tubes at 4° C., 3000 rpm/min, centrifuged for 10 min, and the supernatant plasma was carefully aspirated and stored at −80° C. Assays were performed by natural thawing at room temperature and plasma sample pre-treatment procedures were followed to determine plasma concentrations in rats following a single dose using the established LC-MS/MS method.

### 5.1.2 Tissue Distribution Kinetics Studies

40 healthy male SD rats, weighing 200-220 g, were housed adaptively for three days and fasted for 12 h prior to the experiment. All rats were randomly divided into 5 groups, 8 for each group. Each group of rats was gavaged with the ADD-16 solution (10 mg/kg), anesthetized at 10, 30, 60, 240, 480 min after administration, and sacrificed by exsanguination of the abdominal aorta, respectively. Each group of rats was sacrificed by dissection to collect tissues such as heart, liver, spleen, lung, kidney, brain, and islets. After washing the tissue samples with physiological saline to clean surface blood, the tissue samples were blotted with filter paper to dry surface moisture, weighed, and stored at −80° C. The assay was performed by natural thawing at room temperature and the concentration of ADD-16 in each tissue was determined using the LC-MS/MS method.

### 5.2 Results

### 5.2.1 Plasma Kinetics Results

As can be seen from FIG. 7, SD rats absorbed ADD-16 solution quickly after oral administration and reached a maximum blood concentration at 30 min. The pharmacokinetic parameters of ADD-16 were calculated using DAS 3.0 statistical software and had a half-life (t_{1/2z}) of about 30.2 h, an apparent volume of distribution (Vz/F) of about 0.36 L/kg, a clearance (CLz/F) of about 0.009 L/h/kg, and a plasma concentration maximum (Cₘₐₓ) of about 395.0 ng/mL, see Table 2.

TABLE 2
Pharmacokinetic Parameters Following Single
Oral Administration of ADD-16 in Rats (n = 8)

| | | Mean |
|---|---|---|
| Parameter | Unit | (x̅ ± s) |
| AUC₍₀₋ₜ₎ | ng/mL · h | 1125.5 ± 35.7 |
| AUC_{(0-∞)} | ng/mL · h | 2424.9 ± 527.2 |
| MRT₍₀₋ₜ₎ | h | 8.8 ± 0.4 |
| MRT_{(0-∞)} | h | 37.5 ± 9.2 |
| VRT₍₀₋ₜ₎ | h\hat{ }² | 55.2 ± 3.0 |
| VRT_{(0-∞)} | h\hat{ }² | 1908.2 ± 835.5 |
| t_{1/2z} | h | 30.2 ± 6.6 |
| Tₘₐₓ | h | 0.5 ± 0 |
| CLz/F | L/h/kg | 0.009 ± 0.002 |
| Vz/F | L/kg | 0.36 ± 0.07 |
| Cz | ng/mL | 29.8 ± 10.3 |
| Cₘₐₓ | ng/mL | 395.0 ± 14.5 |

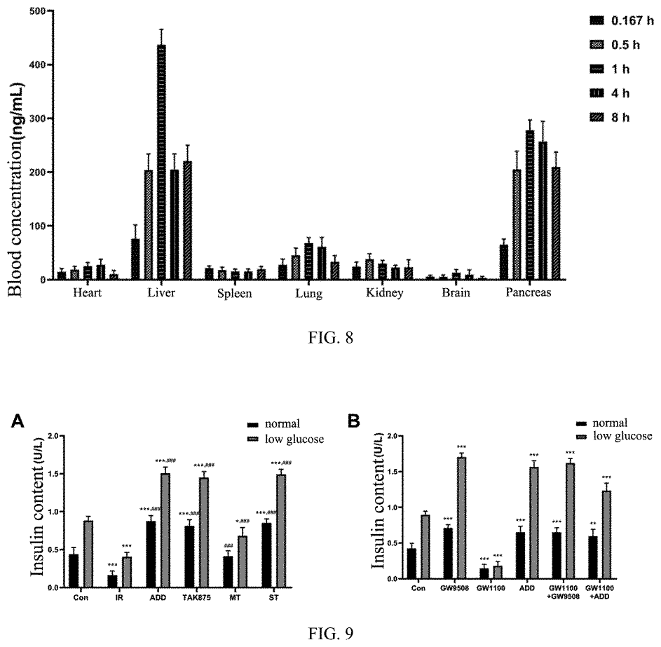

5.2.2 Referring to FIG. 8, the tissue distribution results show that ADD-16 concentrations in other tissues except liver, islets are much lower than blood drug concentrations, Cₘₐₓ order: liver>islets>lung>kidney>heart>spleen>brain, and AUC 0-8 order is consistent with Cₘₐₓ order. The medicament has the lowest concentration in brain tissue, indicating that ADD-16 does not readily penetrate the blood-brain barrier. The medicament has the highest concentration in liver, indicating that the liver may be the major organ for ADD-16 metabolism. GPR40 is predominantly expressed in islet β-cells, and tissue distribution experiments indicate that ADD-16 has well-defined islet targeting.

### 6. Molecular Mechanism Studies of ADD-16 Action Against T2DM

6.1 Methods: MIN6 cells were seeded in 24-well plates and cultured as described in step 2.1 to a cell confluency of more than 80% and administrated in the following groups, with 12 replicates per group: (1) normal control group (Con): cultured with blank 1640 medium; (2) insulin resistance model panel (IR): treated with 0.125 mmol/L PA for 24 h to induce establishment of an insulin resistance model; (3) ADD-16 administration group (ADD): 10 μmol/L ADD-16 intervention was administered for 24 h after successful establishment of the insulin resistance model; (4) TAK875 control group (TAK875): 10 μmol/L TAK875 was administered as a positive control drug intervention for 24 h after successful establishment of the insulin resistance model; (5) metformin control group (MT): 10 mmol/L metformin was administered as a positive control drug intervention for 24 h after successful establishment of the insulin resistance model; (6) sitagliptin control group (ST): 10 μmol/L sitagliptin was administered as a positive control drug intervention for 24 h after successful establishment of the insulin resistance model. After the group administration, the medium was aspirated and washed 2 times with a KRB buffer without sugar, followed by the addition of the KRB buffer without sugar. After incubation at 37° C. for 30 min, the buffer was aspirated and each group was divided into two, i.e., 6 replicates for each group, and KRB buffers containing 2.8 mmol/L and 16.7 mmol/L glucose was added respectively. After incubation at 37° C. for 1 h, all the medium in the wells was aspirated and centrifuged at 2000 rmp/min for 20 min, and the supernatant was collected and stored at −20° C. The insulin content was measured for each group.

MIN6 cells were seeded in 24-well plates and cultured as described in step 2.1 to a cell confluency of more than 80% and administrated in the following groups, with 12 replicates per group: (1) normal control group (Con): cultured with blank 1640 medium; (2) GW9508 group: 1 μmol/L GW9508 intervention for 24 h; (3) GW1100 group: 10 μmol/L GW1100 intervention for 24 h; (4) ADD-16 group (ADD): 10 μmol/L ADD-16 intervention for 24 h; (5) GW1100+GW9508 group: 10 μmol/L GW1100+1 μmol/L GW9508 intervention for 24 h; (6) GW1100+ADD-16 group: 10 μmol/L GW1100+10 μmol/LADD-16 intervention for 24 h. After the group administration, the medium was aspirated and washed 2 times with a KRB buffer without sugar, followed by the addition of the KRB buffer without sugar. After incubation at 37° C. for 30 min, the buffer was aspirated and each group was divided into two, i.e., 6 replicates for each group, and KRB buffers containing 2.8 mmol/L and 16.7 mmol/L glucose was added respectively. After incubation at 37° C. for 1 h, all the medium in the wells was aspirated and centrifuged at 2000 rmp/min for 20 min, and the supernatant was collected and stored at −20° C. The insulin content was measured for each group.

MIN6 cells were seeded in culture dishes at the appropriate density and cultured as described in step 2.1 to a confluency of about 80%. Cells were divided into the following 5 groups: (1) normal control group (Con): cultured with blank 1640 medium; (2) insulin resistance model panel (IR): treatment with 0.125 mmol/L PA for 24 h to induce establishment of a MIN6 cell insulin resistance model; (3) 3 μmol/L ADD-16 administration group (ADD I): 3 μmol/L ADD-16 intervention was given for 24 h after successful establishment of the insulin resistance model; (4) 10 μmol/L ADD-16 administration group (ADD II): 10 μmol/L ADD-16 intervention was given for 24 h after successful establishment of the insulin resistance model; (5) TAK875 control group (TAK875): 10 μmol/L TAK875 was administered as a positive control drug intervention for 24 h after successful establishment of the insulin resistance model. At the end of the group administration, groups of cells were harvested for WB or IF experiments.

### 6.2 Results

### 6.2.1 ADD-16 Ameliorates MIN6 Cell Insulin Resistance

Basal insulin secretion and high glucose-stimulated insulin secretion levels of cells in the IR group were significantly reduced compared to the Con group (P<0.001), while the administration group was able to significantly improve this inhibition phenomenon, the levels of insulin secretion in ADD, TAK875 and ST groups were even higher than in Con group except for MT group (P<0.001), suggesting that ADD-16, TAK875 and sitagliptin not only improved insulin secretion inhibited by insulin resistance, but also stimulated MIN6 cells to secrete more insulin. At the same administration concentrations, the insulinotropic effect of ADD-16 was slightly better than TAK875, see FIG. 9A. ADD-16 was also able to increase insulin secretion in MIN6 cells treated with the GPR40 inhibitor GW1100, with similar effect as GPR40 agonist GW9508, suggesting that compound ADD-16 was able to exert insulinotropic effect by activating GPR40 protein, see FIG. 9B.

### 6.2.2 ADD-16 Promotes Insulin Signaling-Related Molecule Expression in Islet Tissue

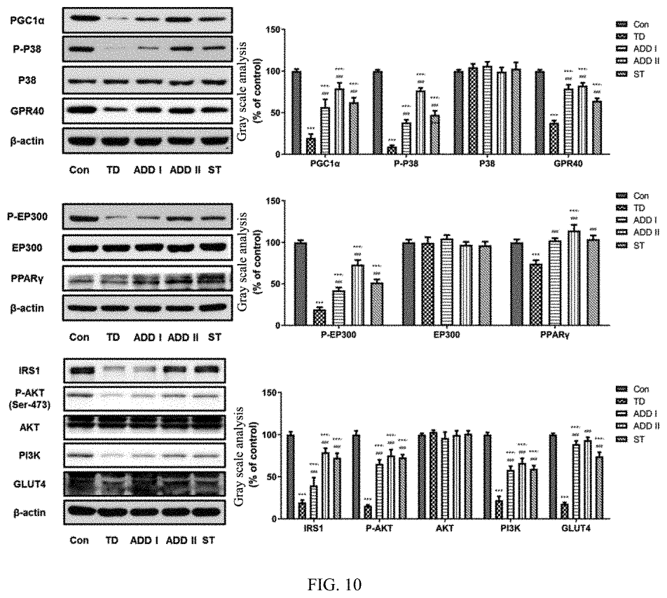

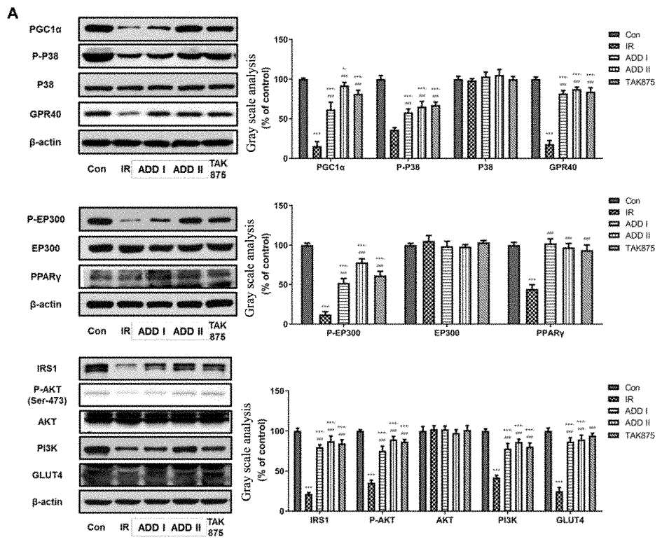

The PI3K/AKT signaling pathway is a classical insulin signaling-related pathway. GPR40 protein activation can induce P38 phosphorylation, which in turn leads to increased expression of PGC-1α. Activation of PGC-1α can promote PPARγ binding to EP300, phosphorylate EP300 and further activate PPARγ, which can activate the PI3K/AKT signaling pathway and induce AKT phosphorylation, stimulate GLUT4 translocation to the cell membrane and increase glucose transport uptake. Referring to FIG. 10, by Western Blot experiments, it was found that ADD-16 was able to upregulate the expression of GPR40, PGC-1α, P-P38, P-EP300, PPARγ, P-AKT, PI3K, IRS1 and GLUT4 in ZDF rat islet tissue compared to TD group (P<0.001), and insulin signaling-related molecule expression changes were most pronounced in the rat islet tissue in the 10 mg/kg administration group.

### 6.2.3 ADD-16 Promotes Insulin Signaling-Related Molecule Expression in MIN6 Cells

After successful improvement of a model of insulin resistance in MIN6 cells, ADD-16 and TAK875 were able to upregulate GPR40, PGC-1α, P-P38, P-EP300, PPARγ, P-AKT, PI3K, IRS1 and GLUT4 expression in MIN6 cells (P<0.001) as measured by Western Blot after 24 h of intervention with different concentrations of ADD-16 and TAK875, with the protein expression changes most pronounced in the 10 μmol/L ADD-16 administration group (see FIG. 11, A). It was also further confirmed by immunofluorescence staining results that ADD-16 was able to upregulate GPR40 expression in MIN6 cells (see FIG. 11B).

### 7. Docking Calculations of Acridinedione Compound to GPR40 Receptor

32 acridinedione compounds were drawn using ChemBioDraw Ultra 14.0 and their structural formulas were introduced into Discovery Studio 2016 to convert the two-dimensional structures of the compounds into three-dimensional space structures. Hydrogen atoms in the formula were replenished using a hydrogenation tool. These 32 compound molecules were superposed according to the base structure (ADD-16) to give MMFF small molecule force field for energy optimization, with parameters set to: intelligent optimization 200 steps.

The GPR40 receptor (PDB id: 4PHU) protein was pretreated with a crystalline complex structure of GPR40 and TAK-875 to remove water molecules from the complex structure and complement non-domain missing amino acids in the protein crystal structure.

Molecular docking was implemented using a semi-flexible molecular docking method based on hot zone matching (LibDock), and the docking operation parameters were: the receptor protein was 4PHU after pre-treatment, ligand molecules were 32 compounds after hydrogenation and energy intelligence optimization, docking area was set to the spatial scale of TAK-875 (radius 13.1808 Angstroms), the number of hot zones was set to 100, the docking decision match threshold was set to 0.25 Angstrom, a high precision docking scoring default algorithm was used, the docking decision decision was selected best, energy optimization of ligand molecules was performed after docking, an intelligent optimization algorithm was selected to improve the accuracy of docking, and the remaining settings remained unchanged. A total of 1925 docking results were obtained, and the highest scoring results for each molecule were summarized and shown in Table 3.

TABLE 3
Docking results of 32 acridinedione compounds with GPR40 molecules

| | | | absolute |
|---|---|---|---|
| No. | Molecular Formula | libdock score | energy |
| 1 | 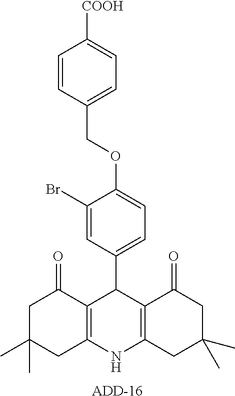 | 147.706 | 40.9335 |
| 2 | 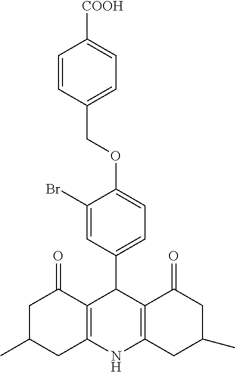 | 149.201 | 42.3492 |
| 3 | 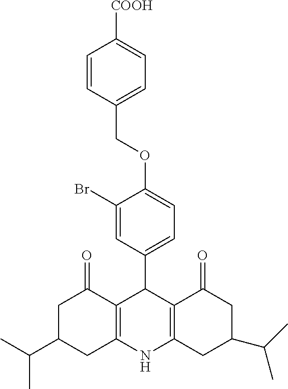 [SMILES: CC(C)C1CC(=O)C2=C(C1)NC1=C(C(=O)CC(C(C)C)C1)C2c1ccc(OCc2ccc(C(=O)O)cc2)c(Br)c1] | 159.056 | 47.7265 |
| 4 | 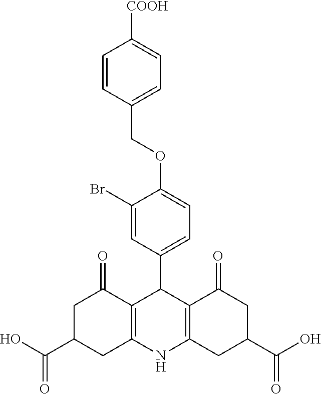 [SMILES: O=C1CC(C(=O)O)CC2=C1C(c1ccc(OCc3ccc(C(=O)O)cc3)c(Br)c1)C1=C(CC(C(=O)O)CC1=O)N2] | 153.023 | 47.9578 |
| 5 | 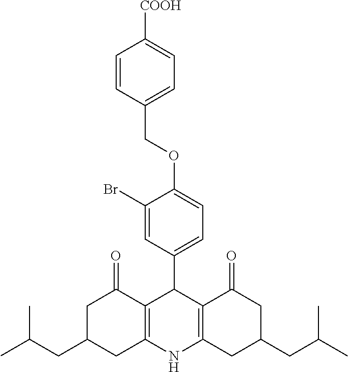 [SMILES: CC(C)CC1CC(=O)C2=C(C1)NC1=C(C(=O)CC(CC(C)C)C1)C2c1ccc(OCc2ccc(C(=O)O)cc2)c(Br)c1] | 161.586 | 47.9627 |
| 6 | 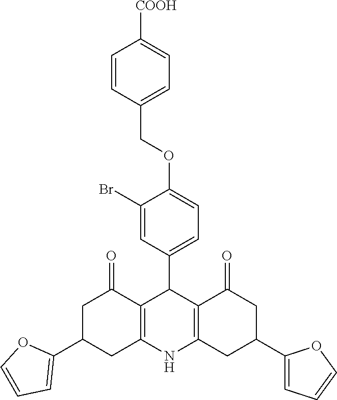 [SMILES: O=C1CC(c2ccco2)CC2=C1C(c1ccc(OCc3ccc(C(=O)O)cc3)c(Br)c1)C1=C(CC(c3ccco3)CC1=O)N2] | 169.537 | 42.4829 |
| 7 | 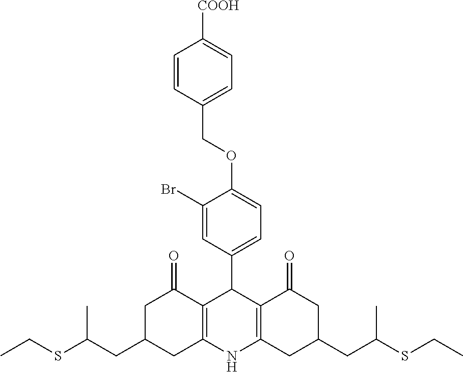 [SMILES: CCSC(C)CC1CC(=O)C2=C(C1)NC1=C(C(=O)CC(CC(C)SCC)C1)C2c1ccc(OCc2ccc(C(=O)O)cc2)c(Br)c1] | 160.574 | 39.8753 |
| 8 | 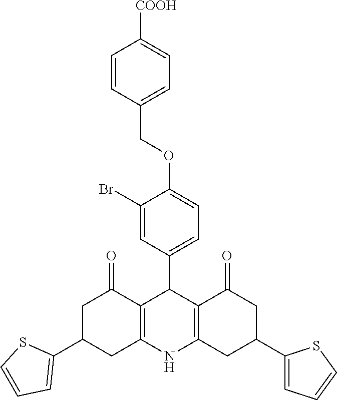 [SMILES: O=C1CC(c2cccs2)CC2=C1C(c1ccc(OCc3ccc(C(=O)O)cc3)c(Br)c1)C1=C(CC(c3cccs3)CC1=O)N2] | 164.633 | 47.5948 |
| 9 | 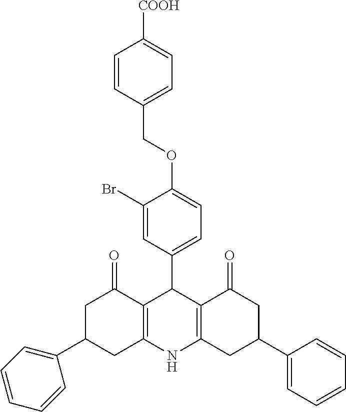 [SMILES: O=C1CC(c2ccccc2)CC2=C1C(c1ccc(OCc3ccc(C(=O)O)cc3)c(Br)c1)C1=C(CC(c3ccccc3)CC1=O)N2] | 175.798 | 72.0423 |
| 10 | 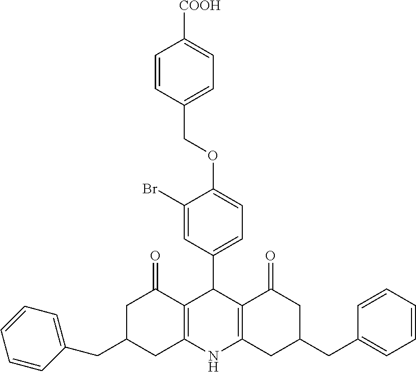 [SMILES: O=C1CC(Cc2ccccc2)CC2=C1C(c1ccc(OCc3ccc(C(=O)O)cc3)c(Br)c1)C1=C(CC(Cc3ccccc3)CC1=O)N2] | 174.513 | 64.1964 |
| 11 | 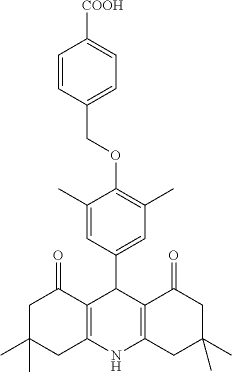 | 148.081 | 44.9332 |
| 12 | 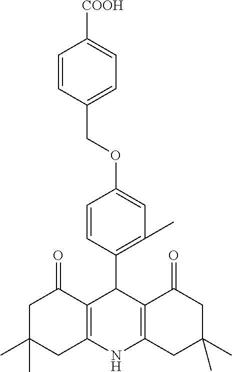 | 147.486 | 45.1903 |
| 13 | 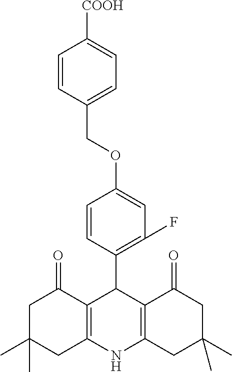 | 153.506 | 47.1667 |
| 14 | 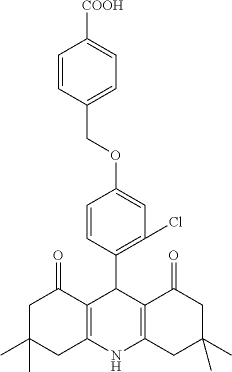 | 151.615 | 42.5155 |
| 15 | 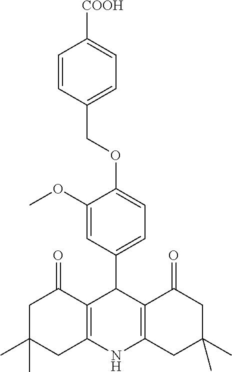 | 154.88 | 43.7166 |
| 16 | 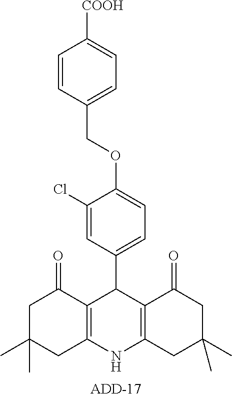 | 149.837 | 41.2808 |
| 17 | 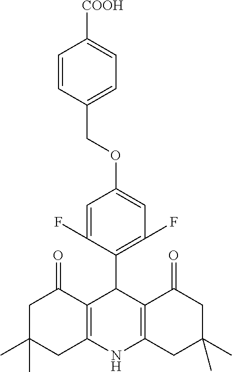 | 151.694 | 43.0447 |
| 18 | 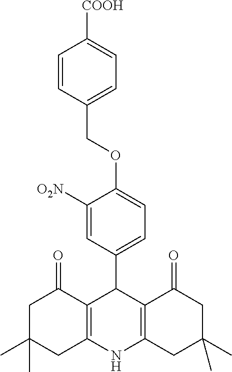 | 153.333 | 43.3727 |
| 19 | 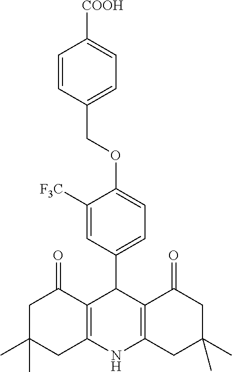 | 144.652 | 51.4433 |
| 20 | 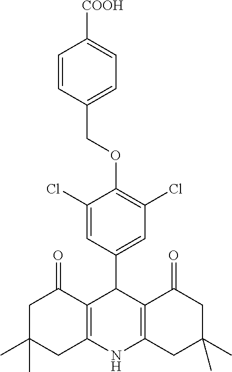 | 145.088 | 47.4351 |
| 21 | 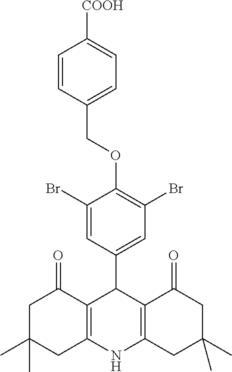 | 144.736 | 49.4108 |
| 22 | 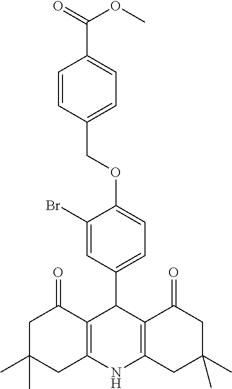 | 152.933 | 44.7276 |
| 23 | 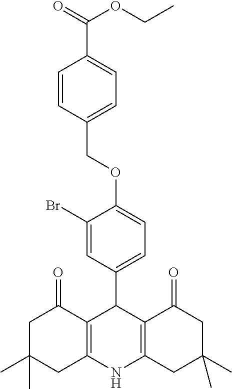 | 145.91 | 40.5961 |
| 24 | 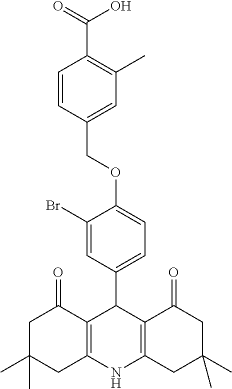 | 155.071 | 52.545 |
| 25 | 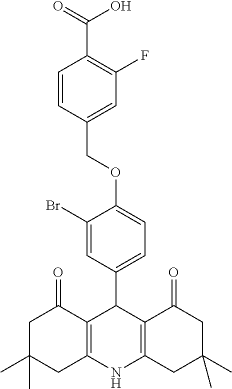 | 158.954 | 41.9706 |
| 26 | 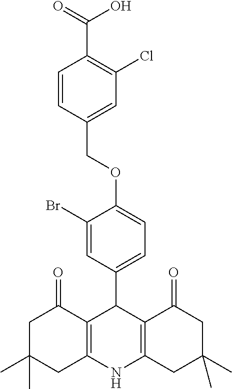 | 161.009 | 44.4609 |
| 27 | 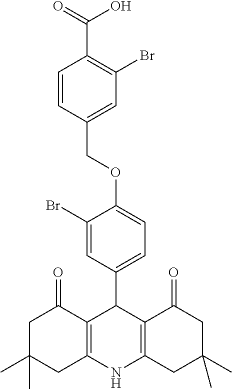 | 156.202 | 55.0304 |
| 28 | 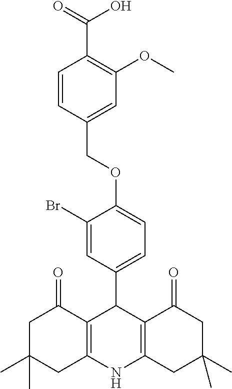 | 156.468 | 53.4514 |
| 29 | 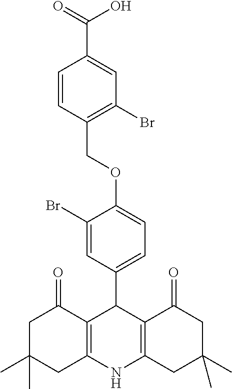 | 147.532 | 48.7284 |
| 30 | 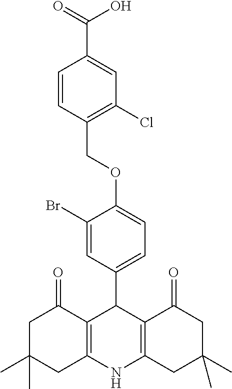 | 150.025 | 49.655 |
| 31 | 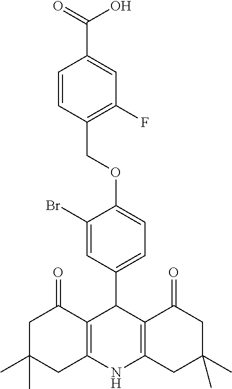 | 151.126 | 42.2519 |
| 32 | 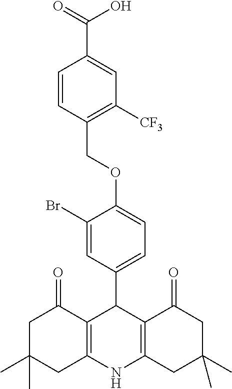 | 151.601 | 53.4345 |

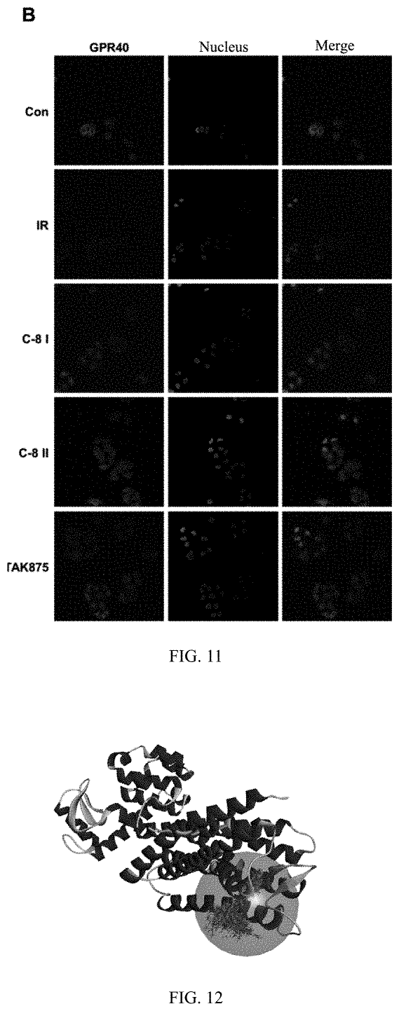

Referring to FIG. 12, it can be seen that the 32 compounds in Table 3 are all capable of binding to GPR40.

Taken together the above experiments, it can be derived that the present application has discovered new drug targets for the acridinedione compound, that is, exerting a glucose-dependent insulinotropic effect by agonizing the GPR40 receptor. The discovery of lead compounds for antidiabetic treatment of such compounds opens up the use of such compounds in drugs against type 2 diabetes, and has important implications for the improvement of antidiabetic medicaments.

See Table 4 for the evaluation of the activity of compounds ADD-16, ADD-17, ADD-18 and ADD-1 of the application.

TABLE 4
Activity Evaluation of GPR40 Agonists (μmol/L, n = 6)

| Compound No. | LD₅₀ | EC₅₀ |
|---|---|---|
| ADD-16 | 123.2 | 12.33 |
| ADD-17 | 156.7 | 20.45 |
| ADD-18 | 107.6 | 11.92 |
| ADD-19 | 122.3 | 13.69 |
| PA | | 6.8 ± 0.5 |

The present application demonstrates for the first time that the acridinedione compound agonizes the GPR40 receptor, participates in the GPR40-PPARγ-PI3K/Akt-GLUT4 signaling pathway, promotes insulin secretion, increases glucose consumption by liver and muscle tissue, improves insulin resistance and exerts an action against type 2 diabetes. The preparation of the acridinedione compound into an antidiabetic medicaments will provide entirely new options and strategies for the treatment of diabetes.

The present application provides an entirely new option and idea for the current treatment of type 2 diabetes, broadening the area of choice of antidiabetic medicaments, and also contributing to the improvement of this technical field. The present application is a compound with a well-defined chemical structure that can be dosed quantitatively for pharmaceutical use, which facilitates the preparation of modern dosage forms and has the potential to be developed into a drug against type 2 diabetes.

1. Use of an acridinedione compound or a pharmaceutically acceptable salt or a pharmaceutically acceptable ester thereof in the preparation of an antidiabetic medicament, wherein, the acridinedione compound has the following structure: 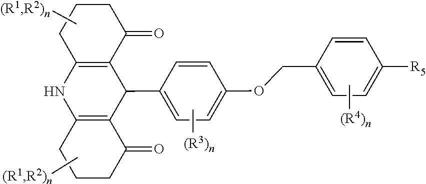
R¹ and R² are each independently hydrogen, C1-C3 alkyl, —COOH, —CH₂Ph, —CH₂CH(CH₃)₂, -Ph, or 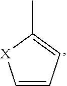
wherein X=O, N or S;
R³ is hydrogen, halogen, —CF₃, C1-C4 alkyl or alkoxy, —NO₂, or —OH;
R⁴ is hydrogen, halogen, —CF₃, C1-C3 alkyl or alkoxy, —NO₂, or —OH;
R⁵ is —COOH, —COOCH₃ or —COOC₂H₅; and
n is 0, 1 or 2.

2. The use of an acridinedione compound or a pharmaceutically acceptable salt or a pharmaceutically acceptable ester thereof in the preparation of an antidiabetic medicament according to claim 1, wherein, the antidiabetic medicament is a GPR40 agonist.

3. The use of an acridinedione compound or a pharmaceutically acceptable salt or a pharmaceutically acceptable ester thereof in the preparation of an antidiabetic medicament according to claim 1, wherein, the antidiabetic medicament is a glucose-dependent insulinotropic drug.

4. The use of an acridinedione compound or a pharmaceutically acceptable salt or a pharmaceutically acceptable ester thereof in the preparation of an antidiabetic medicament according to claim 1, wherein, the antidiabetic medicament is a clinically acceptable pharmaceutical formulation.

5. The use of an acridinedione compound or a pharmaceutically acceptable salt or a pharmaceutically acceptable ester thereof in the preparation of an antidiabetic medicament according to 4, wherein, the pharmaceutical formulation is a tablet, capsule, granule or injection.

6. Use of an acridinedione compound or a pharmaceutically acceptable salt or a pharmaceutically acceptable ester thereof in the preparation of a GPR40 agonist, wherein, the compound has the following structure: 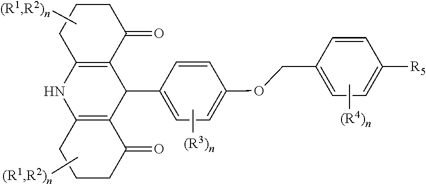
R¹ and R² are each independently hydrogen, C1-C3 alkyl, —COOH, —CH₂Ph, —CH₂CH(CH₃)₂, -Ph, or 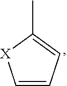
wherein X=O, N or S;
R³ is hydrogen, halogen, —CF₃, C1-C4 alkyl or alkoxy, —NO₂, or —OH;
R⁴ is hydrogen, halogen, —CF₃, C1-C3 alkyl or alkoxy, —NO₂, or —OH;
R⁵ is —COOH, —COOCH₃ or —COOC₂H₅; and
n is 0, 1 or 2.
